(19) United States
(12) Patent Application Publication
(10) Pub. No.: US 2014/0066664 A1
(43) Pub. Date: Mar. 6, 2014

(54) PROCESS FOR THE PREPARATION OF MENTHOL

(71) Applicant: LANXESS Deutschland GmbH, Cologne (DE)

(72) Inventors: Martin MECHELHOFF, Koeln (DE); Claus DREISBACH, Leichlingen (DE); Joerg-Dietrich JENTSCH, Leverkusen (DE); Lutz HEUER, Dormagen (DE); Peter WASSERSCHEID, Erlangen (DE); Anne RITTSTEIGER, Erlangen (DE)

(73) Assignee: LANXESS Deutschland GmbH, Cologne (DE)

(21) Appl. No.: 14/013,412

(22) Filed: Aug. 29, 2013

(30) Foreign Application Priority Data
Aug. 31, 2012 (EP) 12182669.7

(51) Int. Cl. C07C 29/56

(52) U.S. Cl. CPC C07C 29/56
USPC 568/829

(57) ABSTRACT

The invention relates to a process for the preparation of 2-isopropyl-5-methylcyclohexanol (menthol) via the hydrogenation of thymol to neomenthol and the subsequent isomerization to give D/L (+/−)-menthol.

The invention relates to a process for the preparation of 2-isopropyl-5-methylcyclohexanol (menthol) via the hydrogenation of thymol to neomenthol and the subsequent isomerization to give D/L (+/−)-menthol (menthol).

2-Isopropyl-5-methylcyclohexanol has three stereogenic centres, therefore giving eight stereoisomers: D,L-menthol, D,L-neomenthol, D,L-isomenthol and D,L-neoisomenthol.

Among the naturally occurring cyclic terpene alcohols, L-menthol, the main constituent of peppermint oil, assumes a special position on account of its cooling and refreshing effect. L-Menthol is therefore used as a fragrance or flavouring and is used in the pharmaceutical industry. It is therefore the most economically important of the menthol stereoisomers. The general aim has therefore been to carry out the hydrogenation through suitable selection of the reaction conditions and the catalysts such that as much D,L-menthol as possible is formed.

Many substance mixtures whose components have only slight differences in boiling point or even form azeotropes can only be separated with difficulty, if at all, by conventional rectification. This applies to the separation of diastereomers of 2-isopropyl-5-methylcyclohexanol, as are typically formed during the hydrogenation of thymol or subsequent work-up steps. In particular, the separation of the diastereomers isomenthol and menthol can only be performed inadequately and with a high input of energy on account of the low relative volatility of the two compounds relative to one another.

The boiling points of D,L-isomenthol (218.6° C. at 1013 hPa; 75 to 78°C. at 3.3 hPa) and D,L-menthol (216.5° C. at 1013 hPa; 75 to 78° C. at 3.3 hPa) are very close to one another. The separation efficiency of a column during the distillative separation of the individual menthol isomers is therefore determined in particular by the ratio of D,L-menthol to D,L-isomenthol. For a high space-time yield of D,L-menthol during the distillative separation, besides an extremely high D,L-menthol content in the mixture to be separated, an extremely low D,L-isomenthol content is therefore also required. The yield of menthol is thus determined for a given distillation column essentially by the starting ratio of D,L-menthol to D,L-isomenthol.

To produce D,L-menthol, it is known to hydrogenate compounds having the carbon skeleton of menthane with at least one double bond and having oxygen substitution in the 3 position, such as, for example, thymol, in continuous processes over fixed catalyst beds with hydrogen and/or to rearrange stereoisomers of menthol over fixed catalyst beds. DE 23 14 813 A1 describes a process for hydrogenating compounds having the carbon skeleton of menthane with at least one double bond and having oxygen substitution in the 3 position over a bed of a cobalt-manganese catalyst at temperatures of 170° C. to 220° C. and a pressure exceeding 25 bar, preferably exceeding 200 bar. In the examples, temperatures of 180° C. to 210° C. and pressures above 200 bar are employed, and a mixture of the eight stereoisomeric menthols is obtained which consists to 59.5 to 59.9% of the racemic D,L-menthol and to 10.6 to 10.8% of D,L-isomenthol. The maximum menthol/isomenthol ratio is 5.7. By modifying the cobalt-manganese catalyst with copper, menthol mixtures with D,L-menthol contents of 57.6% and D,L-isomenthol contents of 9.2% were achieved, which corresponds to a menthol/isomenthol ratio of about 6.3. The resulting mixtures, however, have 4 to 5% of undesirable by-products in the form of non-reutilizable hydrocarbons.

EP 0 563 611 A 1 and DE 197 18 116 A 1 disclose that the hydrogenation of aromatic or partly hydrogenated cyclic compounds having the carbon skeleton of menthane with at least one C═C double bond and having oxygen substitution in the 3 position can be performed with hydrogen over a fixed bed catalyst comprising palladium, ruthenium or rhodium or a mixture of these elements as active constituents and alkali metal hydroxides and/or sulphates as promoters, in each case applied to a support, the support being doped with a metal from the rare earths and manganese. In the examples, temperatures of 180 to 240° C. and pressures of 270 to 300 bar were employed. Here, menthol mixtures were obtained which forms approx. 52 to 57% D,L-menthol and 11.5 to 14.8% D,L-isomenthol, which corresponds to a menthol/isomenthol ratio of 3.6 to 4.4.

EP 743 296 A 1 discloses catalysts which consist of support-free, compressed powders of cobalt oxides or hydroxides, manganese oxides or hydroxides and alkaline earth metal oxides or hydroxides, and are used at temperatures of 150° C. to 230° C. and pressures of 25 to 350 bar.

The rearrangement of stereoisomers of 1-menthol is described in U.S. Pat. No. 5,756,864: At temperatures of from 200 to 350° C. and hydrogen pressures of 50 to 350 bar, preferably 100 to 300 bar, D-menthol is racemized and isomerized in a continuous process over a catalyst, where the catalyst consists of support-free, compressed powders of nickel hydroxides or oxides, manganese hydroxides or oxides and alkaline earth metal hydroxides or oxides. Here, menthol mixtures were obtained which consisted to a maximum of 59.8% of D,L-menthol.

U.S. 2,843,636 discloses carrying out the isomerization of stereoisomers of menthol to give D,L-menthol with hydrogen in the presence of a hydrogenation catalyst from the group copper chromite, cobalt and nickel at 260 to 280′C and 500 to 1300 p.s.i.g. (34 to 90 bar) in autoclaves. As well as approx. 10 to 12% D,L-isomenthol, the resulting mixtures have a D,L-menthol content of 60 to 64%.

DE 198 53 562 A describes a low-pressure hydrogenation of thymol over a stationary catalyst bed having a temperature gradient: The first two of five serially connected tubular reactors are heated to 180° C., the three tubular reactors behind being heated to 80 to 90° C. Using a catalyst which contains, on a support doped with a metal from the rare earths and with manganese, ruthenium as active constituent and alkali metal hydroxides as promoters, it was possible, at a pressure of 3 bar, to obtain a menthol isomer mixture which comprised 64.4% by weight menthol and 12.1% isomenthol, which corresponds to a menthol/isomenthol ratio of 5.3. Isomerization of a hydrogen-saturated mixture of D,L-neomenthol, D,L-isomenthol and D,L-menthol produced, at atmospheric pressure, an isomer mixture with a composition of 65.3% D,L-menthol and 12.1% isomenthol. In this low-pressure process, it is possible to achieve high menthol contents of approx. 65%. The maximum menthol/isomenthol ratio, however, is 5.4.

DE 100 23 283 A now discloses an improved process in which isomer mixtures which typically have about 55% D,L-menthol can prepare, by means of isomerization with simple supported ruthenium catalysts, menthol-richer mixtures which have up to 67.3% D,L-menthol and only 8.2% D,L-isomenthol, i.e. a menthol/isomenthol ratio of up to 8.1. Furthermore, DE 100 23 283 A discloses that the catalysts can be regenerated with alcoholates, oxides and hydroxides of the alkali metals or alkaline earth metals.

Accordingly, a common aspect of all of the known processes is that they only permit a maximum fraction of around 60% of D,L-menthol, produce at least 8.2% D,L-isomenthol and permit maximum menthol/isomenthol ratios of 8.1.

It was therefore an object of the invention to provide a selective and technically simple process for the preparation of D,L-menthol in high yields, in which ideally no or only small amounts of D,L-isomenthol are formed and which permits high menthol/isomenthol ratios, with the formation of undesired by-products largely being avoided at the same time.

Surprisingly, the object was able to be achieved by a single-stage hydrogenation in which firstly thymol is hydrogenated to a mixture of menthol and neomenthol

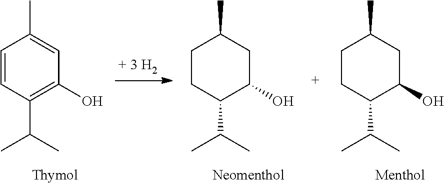

and, after distillative separation, the resulting neomenthol is isomerized by means of catalyst to give D,L-menthol.

The present invention provides a process for the preparation of 2-isopropyl-5-methylcyclohexanol (menthol), according to which

a) thymol is hydrogenated with hydrogen in the presence of a rhodium catalyst, optionally in the presence of a solvent,

b) the resulting neomenthol is separated off from Dl-menthol and

c) the neomenthol isolated in h) is converted to D,L-menthol by means of at least one ruthenium-containing catalyst.

In a preferred embodiment of the present invention, in step a), a 2 to 150-fold excess of hydrogen is used per 1 mol of thymol.

The rhodium-containing catalysts are preferably metallic rhodium and oxidized species of rhodium on the support aluminium oxide.

In a particularly preferred embodiment of the present invention, the rhodium-containing catalysts in part step a) are used in the form of supported or unsupported catalysts.

The support material preferably has a BET surface area of at least 100 m²/g, preferably at least 160 m²/g, particularly preferably at least 180 m²/g. Particular preference is given to aluminium oxide which additionally has a high fraction of macroporous pores with a pore diameter of at least 50 nm and has a pore volume of at least 300 mm³/g, preferably at least 600 mm³/g.

The fraction of rhodium-containing catalyst based on the support material is preferably 0.3-10% by weight, particularly preferably 2-5% by weight,

Very particular preference is given to a support material made of Al₂O₃ and silica with a fraction of 2-5% by weight rhodium.

The catalysts are standard commercial catalysts which are obtainable for example from Heraeus Materials Technology GmbH & Co. KG or Johnson Matthey Plc.

In a preferred embodiment of the process according to the invention, the hydrogenation in part step a) is carried out at temperatures of from 60° -250° C., preferably from 60° -200° C., particularly preferably 60-120° C. and at a pressure of at least 1.1 bar, preferably >1.1 to 325 bar, particularly preferably 2-100 bar.

In a further preferred embodiment of the invention, the hydrogenation in part step a) is carried out in a solvent.

Preferred solvents are cyclic, branched and unbranched alcohols having 1-10 carbon atoms, aliphatic and cyclic ethers having 4-12 carbon atoms and/or aliphatic and cycloaliphatic hydrocarbons having 5-12 carbon atoms, particularly preferably methanol, ethanol, propanol, isopropanol, isobutanol, tetrahydrofuran, diethylene glycol dimethyl ether, ethylene glycol dimethyl ether, tetrahydrofuran, 1,4-dioxane, cyclohexane, methylcyclohexane, cyclooctane, hexane, heptane and/or petroleum ether.

Very particular preference is given to cyclohexane.

The ratio of thymol to solvent is preferably 1: 0 to 1: 20.

In a further embodiment of the invention, the catalyst used in step a) can be recycled. For this purpose, preference is given to using continuous through-flow reactors such as e.g. fluidized-bed reactors or reactors with a fixed catalyst bed.

The neotnenthol formed in step b) is preferably separated off by distillation at temperatures of 60 to 150° C. The distillation bottom, consisting of primarily neomenthol and menthol, is preferably returned to part step a).

The subsequent isomerization of the isolated neomenthol to give D,L-menthol takes place by means of at least one ruthenium-containing catalyst in the form of a supported or unsupported catalyst.

For this purpose, the isomerization catalysts based on ruthenium and optionally alkaline earth metal alkoxylate described in WO2012/010695 are used, which are applied to a support material made of aluminium oxide. Particular preference is given to the use of ruthenium or ruthenium oxides on a support material made of aluminium oxide.

In one preferred embodiment of the process according to the invention, the isomerization c) is carried out at temperatures of from 60° -250′C, preferably from 60° -200° C., particularly preferably 60-150° C. and at a pressure of at least 1.1 bar, preferably >1.1 to 325 bar, particularly preferably 2-100 bar.

Preferred alkaline earth metal alkoxylates are compounds of the formula (I).

(R—O)₂M (I),

in which

R is in each case independent but preferably identical, and is a primary, secondary or tertiary, cyclic or acyclic, branched or unbranched C₁ to C₂₀-alkyl radical which can optionally be further substituted by aryl, C₁-C₄-alkoxyl or C₆ to C₁₄-aryloxy and is particularly preferably methyl, ethyl, n-propyl, isopropyl, n-butyl, sec.-butyl, tert. -butyl, n-pentyl, neopentyl, n-hexyl, cyclohexyl or the stereoisomeric methyl radicals and

M is calcium, strontium or barium, preferably barium.

The preferred barium alkoxylates can be obtained for example by reacting barium perchlorate with the corresponding potassium alkoxylates, preferably dissolved in the same alcohol or a different alcohol, whereupon sparingly soluble potassium perchlorate is formed which can be removed easily from the reaction solutions for example by filtration.

Barium mentholates are for example also obtainable by admixing barium ethoxide or barium isopropoxide with an excess of menthol stereoisomers and prolonged standing or heating.

Particular preference is given to using barium ethoxide, 10% w/v in ethanol, barium isopropoxide as a solid substance, dissolved in menthol isomers, or barium isopropoxide, 20% w/v in isopropanol.

The aluminium oxide used as the support material can be used in all known modifications, preferably in the y modification. The aluminium oxide used as support material advantageously has a BET surface area of at least 100 m²/g, preferably at least 160 m²/g, particularly preferably at least 180 m²/g. Particular preference is given to aluminium oxide which additionally has a high fraction of macroporous pores with a pore diameter of at least 50 nm and has a pore volume of at least 300 mm³/g, preferably at least 600 mm³/g. Examples of suitable support materials include the commercially available aluminium oxides SPH 1515, SPH 531, SPH 501 from Rhodia, D 10-10 from BASF and SA 6176 from Norton.

The support material can be used for example in the form of powders with particle sizes of from 0.001 to 0.1 mm, crushed and sieved material with particle sizes between 0.05 and 5 mm or in mouldings, preferably extrudates, pellets, beads or granules with diameters of from 0.2 to 30 mm.

The particular advantage of the process according to the invention is that mixtures of diastereomers of 2-isopropyl-5-methylcyclohexanols can be separated in an efficient manner such that the diastereomers neomenthol and D,L-menthol are obtained with high purities.

In the process according to the invention, the specific energy consumption can be considerably lowered and the dimensions of the separation apparatuses used, i.e. the required apparatus volume per required separation stage, can be considerably reduced.

The scope of the invention encompases all of the above and below general or preferred radical definitions, indices, parameters and explanations with one another, i.e. also between the respective ranges and preferred ranges in any desired combination.

The examples which follow serve to illustrate the invention but have no limiting effect.

### EXAMPLE 1

6 g of thymol (40 mmol) were hydrogenated with an at least 3 molar excess of hydrogen in the presence of 1 mol % of catalyst (see table below) at temperatures of 120° C. and at a pressure of 30 bar in the presence of cyclohexane as solvent to give neomenthol (Neo).

| | Neomenthol | | Reaction |
|---|---|---|---|
| Catalysts | fraction/% | Neo/Iso | time t/h |
| 5% Rh/Alox, Merck KGaA | 49.0 | 15.5 | 1.50 |
| (0.5 mol %) | | | |
| 5% Rh/activated Alox, Aldrich | 39.7 | 11.1 | 0.25 |
| (0.5 mol %) | | | |

Where Aldrich = Sigma-Aldrich Chemie GmbH and Neo/Iso = neomenthol/isomenthol and Alox = aluminium oxide

Neomenthol was separated off by distillation at a bottom temperature of 142° C. The two catalysts exhibited, with roughly 40% and 49%, very high neomenthol selectivities. The

Nealso ratio in these experiments, at 11.1 (5% Rh/activated Alox, Aldrich) and 15.5 (5% Rh/Alox, Merck KgaA), was also very high. For an industrial application, however, not only are the selectivity and the Neo/Iso ratio decisive, but also the reaction time, The ratio 11.1 was achieved after just 25 min, and 15.5 after just 1.5 h.

For the isomerization, neomenthol was reacted with 1 mol % RuO₂ in cyclohexane at temperatures of 75° C. and 100° C. and a pressure of 10 bar.

| Temperature | | Fraction of | Fraction of D,L- |
|---|---|---|---|
| T/° C. | Time t/h | neomenthol/% | menthol/% |
| 75° C. | 22 h | 32 | 68 |
| 100° C. | 7 h | 33 | 67 |

At a reaction temperature of 100° C., the thermodynamic equilibrium between neo- and D,L-menthol could be achieved after, at the latest, a reaction time of 7 hours. Here, fewer than 1% of negligible amounts of by-products and other menthol isomers were formed.

The concentration data refer to gel chromatography area %.

### EXAMPLE 2

### Comparative Example

6 g of thymol (40 mmol) were hydrogenated with an at least 3 molar excess of hydrogen in the presence of 1 mol % of catalyst (see table below) at temperatures of 120° C. and at a pressure of 30 bar in the presence of cyclohexane as solvent to give neomenthol (Neo).

| | Neomenthol | | |
|---|---|---|---|
| Catalysts | fraction/% | Neo/Iso | Reaction time t/h |
| 5% Rh/Alox, (0.5 mol %) | 28 | 2.8 | 50 |

For the isomerization, the resulting mixture was reacted with 1 mol % of RuO2 in cyclohexane at temperatures of 75° C. or 100° C. and a pressure of 10 bar.

| Temperature | | Fraction of | Fraction of D,L- |
|---|---|---|---|
| T/° C. | Time t/h | neomenthol/% | menthol/% |
| 100° C. | 7 h | 28 | 62 |

The fraction of isomenthol was 10%. On account of the boiling points of isomenthol and D,L-menthol being close together, a separation is possible only with approximately 1.6-fold energy consumption, which renders the entire process economically unattractive.

The concentration data refer to gel chromatography area %.

What is claimed is:

1. Process for the preparation of 2-isopropyl-5-methylcyclohexanol (menthol), characterized in that
a) thymol is hydrogenated with hydrogen in the presence of a rhodium catalyst, optionally in the presence of a solvent,
b) the resulting neomenthol is separated off from menthol and the neomenthol isolated in b) is converted to menthol by means of at least one ruthenium-containing catalyst in supported or unsupported form.

2. Process according to claim 1, characterized in that hydrogenation in stage a) is carried out at temperatures of from 60′ to 250° C., preferably from 60°-200° C., particularly preferably 60-120° C. and at a pressure of at least 1.1 bar, preferably >1.1 to 325 bar, particularly preferably 2-100 bar.

3. Process according to claim 1 or 2, characterized in that supported catalysts, preferably catalysts supported on Al₂O₃, are used as ruthenium catalysts.

4. Process according to one or more of claims 1 to 3, characterized in that cyclic, branched and unbranched alcohols having 1-10 carbon atoms, aliphatic and cyclic ethers having 4-12 carbon atoms and/or aliphatic and cycloaliphatic hydrocarbons having 5-12 carbon atoms, preferably methanol, ethanol, propanol, isopropanol, isobutanol, tetrahydrofuran, diethylene glycol dimethyl ether, ethylene glycol dimethyl ether, tetrahydrofuran, 1,4-dioxane, cyclohexane, methylcyclohexane, cyclooctane, hexane, heptane and/or petroleum ether, are used as solvents.

5. Process according to one or more of claims 1 to 4, characterized in that step c) is carried out at temperatures of from 60°-250° C., preferably from 60°-200° C., particularly preferably 60-120° C., and at a pressure of at least 1.1 bar, preferably >1.1 to 325 bar, particularly preferably 2-100 bar.
